(19) United States
(12) Patent Application Publication
(10) Pub. No.: US 2026/0174663 A1
(43) Pub. Date: Jun. 25, 2026

(54) EXTERNAL PREPARATION FOR SKIN

(71) Applicant: SHISEIDO COMPANY, LTD., Tokyo (JP)

(72) Inventors: Haruhiko INOUE, Tokyo (JP); Shizuma TANAKA, Tokyo (JP)

(73) Assignee: SHISEIDO COMPANY, LTD., Tokyo (JP)

(21) Appl. No.: 19/128,677

(22) PCT Filed: Nov. 24, 2023
(86) PCT No.: PCT/JP2023/042107

§ 371 Date: May 9, 2025

(30) Foreign Application Priority Data
Dec. 9, 2022 (JP) 2022-197315

(51) Int. Cl. A61K 8/67; A61K 8/37; A61K 8/92; A61Q 19/08

(52) U.S. Cl. CPC A61K 8/671; A61K 8/375; A61K 8/922; A61Q 19/08

(57) ABSTRACT

The present invention provides an external preparation for skin that produces a wrinkle-reducing effect at an early stage after application and maintains the effect for an extended period. An external preparation for skin according to the present invention contains (A) retinol, (B) castor oil, (C) bis-diglyceryl polyacyladipate-2 or dipentaerythrityl hexahydroxystearate, and (D) water.

### TECHNICAL FIELD

The present invention relates to an external preparation for skin having a high wrinkle-reducing effect when applied to skin.

### BACKGROUND ART

Retinol is known to be an effective component for prevention and treatment of, for example, skin keratosis, and prevention or recovery of skin aging, and is used in cosmetics that are expected to have a wrinkle-reducing effect. However, it is desired to exhibit the wrinkle-reducing effect at an earlier stage. Since retinol is likely to undergo, for example, isomerization, decomposition, or polymerization due to light, air, heat, metal ions, or the like, further improvement of retinol blending technology is desired from the viewpoint of retinol stability.

In addition, it is known that softening the stratum corneum of the skin is effective in realizing the wrinkle-reducing effect. Although an external preparation for skin for softening the stratum corneum is also known, further modification is desired from the viewpoint of the wrinkle-reducing effect and stability.

From such a background, an external preparation for skin that produces a wrinkle-reducing effect at an early stage after application and maintains the effect for an extended period is desired.

### CITATION LIST

### Patent Literature

Patent Literature 1: JP H6-024958 A

### SUMMARY OF THE INVENTION

The present invention has been completed in view of the problems as described above, and an object of the present invention is to provide an external preparation for skin that produces a wrinkle-reducing effect at an early stage and has excellent temporal stability.

According to the present invention, the following invention is provided.

[1]

An external preparation for skin, comprising:
(A) retinol,
(B) castor oil,
(C) bis-diglyceryl polyacyladipate-2 or dipentaerythrityl hexahydroxystearate, and
(D) water.

[2]

The external preparation for skin according to [1], which is an oil-in-water emulsified preparation.

[3]

The external preparation for skin according to [1] or [2], wherein a content of the component (A) is 0.005 to 5% by mass based on a total mass of the external preparation for skin.

[4]

The external preparation for skin according to any one of [1] to [3], wherein a content of the component (B) is 0.2 to 30% by mass based on a total mass of the external preparation for skin.

[5]

The external preparation for skin according to any one of [1] to [4], wherein a content of the component (C) is 0.2 to 30% by mass based on a total mass of the external preparation for skin.

[6]

The external preparation for skin according to any one of [1] to [5], wherein a total content of the component (B) and the component (C) is 0.5 to 40% by mass based on a total mass of the external preparation for skin.

[7]

The external preparation for skin according to any one of [1] to [6], wherein a ratio of [total amount of component (B) and component (C) contained]/[amount of component (A) contained] is 10 to 500.

[8]

The external preparation for skin according to any one of [1] to [7], wherein the component (B) is an ester of a fatty acid having 14 to 20 carbon atoms and glycerin.

[9]

The external preparation for skin according to any one of [1] to [8], further comprising a hydrogenated lecithin (E).

[10]

The external preparation for skin according to any one of [1] to [9], further comprising a higher alcohol (F).

[11]

The external preparation for skin according to any one of [1] to [10], further comprising a polyhydric alcohol (G).

### Advantageous Effects of the Invention

According to the present invention, there is provided an external preparation for skin that produces a wrinkle-reducing effect at an early stage after being applied to the skin while maintaining the effect, i.e., having excellent stability of the effect.

### DETAILED DESCRIPTION OF THE INVENTION

### [External Preparation for Skin]

An external preparation for skin according to the present invention contains essential components:
(A) retinol,
(B) castor oil,
(C) bis-diglyceryl polyacyladipate-2 or dipentaerythrityl hexahydroxystearate, and
(D) water. Hereinafter, these essential components and additional components that can be used in combination will be described.

### [(A) Retinol]

An external preparation for skin according to the present invention contains retinol (hereinafter, sometimes referred to as a component (A)). Retinol is commonly used in the fields of cosmetics, pharmaceuticals, quasi-drugs, and the like, which can be arbitrarily selected even in the present invention.

Retinol (also called vitamin A) has several stereoisomers such as all-trans type, 13-cis type, and 9 cis type, all of which can be used.

An amount of retinol (component (A)) blended is not particularly limited as long as the amount is effective for exhibiting a wrinkle-reducing effect, but is usually preferably 0.005 to 5% by mass and more preferably 0.01 to 1% by mass based on the total mass of the external preparation for skin. When an amount of retinol contained is within such a range, a sufficient wrinkle-reducing effect can be obtained.

### [(B) Castor Oil]

An external preparation for skin according to the present invention contains castor oil (hereinafter, sometimes referred to as a component (B)). Castor oil is an ester of fatty acids and glycerin extracted from the seeds of the castor-oil plant belonging to the family Euphorbiaceae as natural fat and oil. The fatty acids constituting castor oil is composed of about 90% of ricinoleic acid, palmitic acid, stearic acid, oleic acid, linoleic acid, linolenic acid, and the like.

Castor oil is generally extracted from natural components, and the water content, coloration, acid value, and the like may slightly vary depending on the purification conditions, but from the viewpoint of quality stability of cosmetics, castor oil having high purity is preferred. In addition, from the viewpoint of storage stability, castor oil having a low acid value, for example, an acid value of 0.5 mg KOH/g or less is preferred.

As the castor oil, a synthesized compound corresponding to natural castor oil can also be used. Specifically, an ester of a fatty acid having 14 to 20 carbon atoms and glycerin can also be used as the component (B).

The content of the castor oil (component (B)) is preferably 0.2 to 30% by mass and more preferably 0.5 to 20% by mass based on the total mass of the external preparation for skin. When an amount of castor oil contained is within such a range, the effects of the present invention can be more strongly exhibited.

[(C) Bis-diglyceryl polyacyladipate-2/dipentaerythrityl hexahydroxystearate]

An external preparation for skin according to the present invention contains bis-diglyceryl polyacyladipate-2 or dipentaerythrityl hexahydroxystearate (hereinafter, sometimes referred to as a component (C)). The component (C), in combination with the component (B), exerts an effect of softening the stratum corneum from an early stage after the external preparation for skin is applied to the skin, and further enhances the wrinkle-reducing effect of the component (A). It is considered that this effect is exhibited because the oxidation of the component (A) is effectively suppressed by the combination of the component (B) and the component (C).

The content of the component (C) is preferably 0.2 to 30% by mass and more preferably 0.5 to 20% by mass based on the total mass of the external preparation for skin. In addition, the total content of the component (B) and the component (C) is preferably 0.5 to 40% by mass, more preferably 0.5 to 30% by mass, and still more preferably 1 to 20% by mass. When the total content of the component (B) and the component (C) is 0.5% by mass or more, a sufficient wrinkle-reducing effect can be achieved, and when the total content is 40% by mass or less, stickiness is suppressed to improve the feeling of use.

The ratio of [total amount of component (B) and component (C) contained]/[amount of component (A) contained] is preferably 10 to 500 and more preferably 10 to 300.

When amounts of the component (B) and the component (C) contained are prepared to satisfy such conditions, the external preparation for skin can exert an effect of softening the stratum corneum from an early stage after being applied to the skin and also produces a wrinkle-reducing effect for an extended period.

### [(D) Water]

The external preparation for skin according to the present invention further contains water in addition to the above components. As the water, water used for cosmetics or quasi-drugs can be used, and for example, purified water, ion-exchanged water, tap water, or the like can be used.

### [Other Components]

The external preparation for skin according to the present invention may contain, in addition to the components (A) to (D) described above, other components, if necessary, as long as the effects of the present invention are not impaired.

One of such other components is hydrogenated lecithin (hereinafter, sometimes referred to as a component (E)). Hydrogenated lecithin is a lecithin derivative obtained by adding hydrogen to lecithin containing phospholipid as a main component. Lecithin has a hydrophilic group composed of a phosphate ester salt-type anionic active group and a quaternary ammonium salt-type cationic active group, and a hydrophobic group having a saturated aliphatic carboxylic acid residue such as palmitic acid or stearic acid and an unsaturated aliphatic carboxylic acid residue such as oleic acid or linoleic acid. Hydrogenated lecithin is obtained by hydrogenating this lecithin to improve the degree of saturation, resulting in improved oxidative stability.

The component (E) is effectively used as a dispersant when the external preparation for skin according to the present invention is made into an emulsified product such as an oil-in-water emulsified preparation. When the component (E) is blended, the content thereof is preferably 0.1 to 3% by mass and more preferably 0.5 to 3% by mass based on the total mass of the external preparation for skin.

Another example of such other components includes a higher alcohol (hereinafter, sometimes referred to as a component (F)). In the present invention, the higher alcohol is not particularly limited as long as it can be used in the fields of cosmetics, pharmaceuticals, quasi-drugs, and the like. Specific examples thereof include stearyl alcohol, behenyl alcohol, lauryl alcohol, myristyl alcohol, and cetyl alcohol. In addition, a mixture of the alcohols, for example, a combination of stearyl alcohol and behenyl alcohol is preferred.

When the component (F) is blended, the content thereof is preferably 0.1 to 20% by mass and more preferably 0.5 to 10% by mass based on the total mass of the external preparation for skin.

Another example of such other components includes a polyhydric alcohol (hereinafter, sometimes referred to as a component (G)). Such a polyhydric alcohol can be arbitrarily selected as long as the effects of the present invention are not impaired. Specifically, the polyhydric alcohol is preferably one or more selected from the group consisting of glycerin, diglycerin, polyglycerin, 1,3-butylene glycol, ethylene glycol, propylene glycol, dipropylene glycol, polyethylene glycol, polypropylene glycol, polyethylene glycol, polypropylene glycol, trimethylolethane, trimethylolpropane, erythritol, pentaerythritol, sorbitan, glucose, sorbitol, maltitol, sucrose, raffinose, hexylene glycol, 1,2-pentanediol, and trehalose.

The component (G) exerts a moisturizing function and a thermal sensation imparting function, and the content thereof is adjusted according to the purpose. Generally, the content of the component (G) is preferably 5 to 40% by mass and more preferably 10 to 30% by mass based on the total mass of the external preparation for skin.

The external preparation for skin according to the present invention may contain components usually used for an external preparation for skin other than the above components. Examples of such other components include powder components, liquid fats and oils, solid fats and oils, waxes, hydrocarbon oils, higher fatty acids, ester oils, silicone oils, anionic surfactants, cationic surfactants, amphoteric surfactants, nonionic surfactants, humectants, water-soluble polymers, film-forming agents, ultraviolet absorbers, sequestering agents, lower alcohols, sugars, amino acids, organic amines, polymer emulsions, pH adjusting agents, skin nutrients, vitamins, antioxidants, antioxidant aids, and fragrances.

The dosage form of the external preparation for skin according to the present invention is preferably an emulsified preparation and may be either an oil-in-water emulsified preparation or a water-in-oil emulsified preparation. However, an oil-in-water emulsified preparation having excellent feeling of use is preferred. In addition, any dosage type such as a liquid preparation, an emulsion preparation, or a cream preparation can be employed.

### EXAMPLES

The present invention will be described below using examples. Note that the present invention is not limited by these examples at all. In addition, the content is mass % with respect to the total amount unless otherwise specified.

### Examples 1 to 4 and Comparative Examples 1 and 2

An external preparation for skin was prepared by blending each component to have the content shown in Table 1. The percent residue of retinol and the stratum corneum-softening effect of the obtained external preparation for skin were evaluated.

Regarding the percent residue of retinol, the obtained external preparation for skin was divided into two parts, and retinol contents after storage at 0° C. and 40° C. for 2 months were each measured, and based on the retinol content of a sample stored at 0° C., the percent retinol residue of a sample stored at 40° C. was evaluated.

Regarding the stratum corneum-softening effect, the stratum corneum peeled off from the human skin was cut out into a size of 20 mm×3 mm and placed in a stratum corneum viscoelasticity measuring instrument, to which 3 μL of the external preparation for skin was then applied, followed by measurement of the storage elastic modulus at a temperature of 32° C. and a humidity of 50%. The measurement conditions were as follows.
Measurement frequency: 1 Hz
Strain: 0.2%

The evaluation criteria for the stratum corneum-softening effect were as follows based on the measured storage elastic modulus value, with the value of Comparative Example 1 using hydrogenated polydecene as an oil component being 100.
S: less than 50
A: 50 or more and less than 60
B: 60 or more and less than 80
C: 80 or more and 100 or less

TABLE 1

| | | Comparative Example | | Example | | | |
|---|---|---|---|---|---|---|---|
| Component | | 1 | 2 | 1 | 2 | 3 | 4 |
| A | Retinol | 0.1 | 0.1 | 0.1 | 0.1 | 0.1 | 0.1 |
| B | Castor oil | | 1 | 1 | 2 | 2 | 2 |
| C | Bis-diglyceryl polyacyladipate-2 | | | 2 | 1.5 | 1 | |
| | Dipentaerythrityl hexahydroxystearate | | | | | | 1 |
| D | Water | balance | balance | balance | balance | balance | balance |
| F | Behenyl alcohol | 0.3 | 0.3 | 0.3 | 0.3 | 0.3 | 0.3 |
| | Stearyl alcohol | 0.1 | 0.1 | 0.1 | 0.1 | 0.1 | 0.1 |
| G | Glycerin | 5 | 5 | 5 | 5 | 5 | 5 |
| | DPG | 5 | 5 | 5 | 5 | 5 | 5 |
| | BG | 7 | 7 | 7 | 7 | 7 | 7 |
| | Hydrogenated polydecene | 2 | | | | | |
| | Ethanol | 4 | 4 | 4 | 4 | 4 | 4 |
| | PEG-20 | 3 | 3 | 3 | 3 | 3 | 3 |
| | Sodium acrylate/sodium acryloyldimethyltaurate | 2.5 | 2.5 | 2.5 | 2.5 | 2.5 | 2.5 |
| | copolymer/water/isohexadecane/polysorbate | | | | | | |
| | 80/sorbitan oleate | | | | | | |
| | PEG-60 hydrogenated castor oil | 0.8 | 0.8 | 0.8 | 0.8 | 0.8 | 0.8 |
| | Limnanthes alba oil | 4 | 4 | 4 | 4 | 4 | 4 |
| | Pentaerythrityl tetraethylhexanoate | 12 | 12 | 12 | 12 | 12 | 12 |
| | Dimethicone | 2 | 2 | 2 | 2 | 2 | 2 |
| | Tocopheryl acetate | 0.5 | 0.5 | 0.5 | 0.5 | 0.5 | 0.5 |
| | Phenoxyethanol | q.s. | q.s. | q.s. | q.s. | q.s. | q.s. |
| | EDTA-2Na | q.s. | q.s. | q.s. | q.s. | q.s. | q.s. |
| | Sodium metabisulfite | q.s. | q.s. | q.s. | q.s. | q.s. | q.s. |
| | BHT | q.s. | q.s. | q.s. | q.s. | q.s. | q.s. |
| | Fragrance | q.s. | q.s. | q.s. | q.s. | q.s. | q.s. |
| | B + C | 0 | 1 | 3 | 3.5 | 3 | 3 |
| | (B + C)/A | 0 | 10 | 30 | 35 | 30 | 30 |
| Evaluation | Percent residue of retinol (%) | 81.9 | 82.7 | 93.4 | 93.9 | 95.5 | 92.1 |
| | Stratum corneum-softening effect | C | B | S | S | A | A |

Hereinafter, other formulation examples of the external preparation for skin according to the present invention will be described.

[Formulation Example 1]

| Purified water | Balance |
|---|---|
| Glycerin | 7 |
| BG | 8 |
| DPG | 7 |
| Carbomer | 0.25 |
| Potassium hydroxide | 0.1 |
| Sodium methyl stearoyl taurate | 1 |
| Hydrogenated lecithin | 1 |
| Behenyl alcohol | 3 |
| Stearyl alcohol | 1 |
| Hydrogenated polydecene | 3 |
| Dimethicone | 5 |
| Castor oil | 5 |
| Bis-diglyceryl polyacyladipate-2 | 2 |
| Retinol | 0.5 |
| Tocopherol acetate | 0.5 |
| EDTA-2Na | 0.02 |
| Phenoxyethanol | 0.2 |
| Total | 100 |

(unit: % by mass)

[Formulation Example 2]

| Purified water | Balance |
|---|---|
| Glycerin | 10 |
| BG | 5 |
| DPG | 5 |
| Carbomer | 0.1 |
| Potassium hydroxide | 0.04 |
| Beheneth-20 | 1 |
| Hydrogenated lecithin | 0.5 |
| Behenyl alcohol | 4 |
| Stearyl alcohol | 1 |
| Hydrogenated polydecene | 3 |
| Dimethicone | 5 |
| Castor oil | 5 |
| Dipentaerythrityl hexahydroxystearate | 1 |
| Retinol | 0.5 |
| Niacinamide | 5 |
| Tocopherol | 0.1 |
| EDTA-2Na | 0.1 |
| Phenoxyethanol | 0.5 |
| Fragrance | 0.1 |
| Total | 100 |

(unit: % by mass)

1. An external preparation for skin, comprising:
(A) retinol,
(B) castor oil,
(C) bis-diglyceryl polyacyladipate-2 or dipentaerythrityl hexahydroxy-stearate, and
(D) water.

2. The external preparation for skin according to claim 1, which is an oil-in-water emulsified preparation.

3. The external preparation for skin according to claim 1, wherein a content of the component (A) is 0.005 to 5% by mass based on a total mass of the external preparation for skin.

4. The external preparation for skin according to claim 1, wherein a content of the component (B) is 0.2 to 30% by mass based on a total mass of the external preparation for skin.

5. The external preparation for skin according to claim 1, wherein a content of the component (C) is 0.2 to 30% by mass based on a total mass of the external preparation for skin.

6. The external preparation for skin according to claim 1, wherein a total content of the component (B) and the component (C) is 0.5 to 40% by mass based on a total mass of the external preparation for skin.

7. The external preparation for skin according to claim 1, wherein a ratio of [total amount of component (B) and component (C) contained]/[amount of component (A) contained] is 10 to 500.

8. The external preparation for skin according to claim 1, wherein the component (B) is an ester of a fatty acid having 14 to 20 carbon atoms and glycerin.

9. The external preparation for skin according to claim 1, further comprising a hydrogenated lecithin (E).

10. The external preparation for skin according to claim 1, further comprising a higher alcohol (F).

11. The external preparation for skin according to claim 1, further comprising a polyhydric alcohol (G).
